(19) United States
(12) Patent Application Publication
(10) Pub. No.: US 2012/0010572 A1
(43) Pub. Date: Jan. 12, 2012

(54) Intravenous Catheter Protective Cover

(76) Inventor: Tionne Bennett, Cerritos, CA (US)

(21) Appl. No.: 13/182,174

(22) Filed: Jul. 13, 2011

Related U.S. Application Data
(63) Continuation-in-part of application No. 11/257,224, filed on Oct. 24, 2005.
(60) Provisional application No. 61/492,442, filed on Jun. 2, 2011.

(51) Int. Cl. A61M 25/02

(52) U.S. Cl. 604/180; 604/174

(57) ABSTRACT

A cover for an intravenous catheter configured to attach to a patient to thereby prevent unintentional jarring or other contact with the catheter. The cover includes a dome configured to receive the catheter, and can include a first aperture through which the catheter can be inserted. A fastener can be coupled to the dome, such that the cover can be fastened to the patient. A first set of detents extends from the dome, which are configured to restrict movement of the catheter with respect to the dome.

This application is a Continuation-In-Part of U.S. application Ser. No. 11/257,224 filed on Oct. 24, 2005. This application also claims priority to U.S. Provisional Application No. 61/492,442 filed on Jun. 2, 2011. These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is intravenous catheters, and more particularly to devices for securing catheters to the location at which the catheter is attached to the patient.

### BACKGROUND

Typically, when a vascular catheter is placed, for example for intravenous administration of fluids, a combined catheter and needle is used to create the vascular puncture after which the needle is removed and the hub of the remaining catheter attached to a source of infusion liquid. Generally, it is necessary to stabilize the catheter in relation to the blood vessel to prevent movement of the catheter to preclude catheter withdrawal, undesirable additional blood vessel punctures and the like. The catheter hub is typically stabilized by taping the hub and associated tube fittings to the patient's skin in the area adjacent the vessel puncture.

Using precautions, such as sterile gloves, while inserting the catheters has made the process of securing these catheters complicated. Indeed, in general, it is difficult to apply sticky tape to the skin while wearing gloves. In addition, the extra movement needed to reach for and to apply such tape risks the loss of the unsecured catheter. This is particularly true where the catheter has been placed in a child. As a result, it is not uncommon for the nurse or medical assistant to be forced to remove the gloves so that the catheter can be secured. Thus, the nurse or medical assistant is left without any protection for himself/herself while attempting to secure the catheter in its proper position.

There have been attempts in the past to address the foregoing problem of stabilization of an inserted needle or catheter immediately following vein puncture.

One approach has been to provide an adhesive surface on the underside of an element bearing wings provided on the catheter hub. Such intravenous needle assemblies are shown, for example, in U.S. Pat. No. 4,324,236 and U.S. Pat. No. 4,627,842. A difficulty encountered with such prior art products, however, is that the protective covering for the adhesive on the wings must be removed before the needle is inserted into the vein so that it may be thereafter adhered to the skin surface. Providing an adhesive surface that is exposed before vein puncture, however, risks premature adhesion to the skin and/or adhesion to the medial care provider's gloves.

Another approach has been to provide an anchoring system, including a device that permits a portion of a catheter or similar medical article to be anchored to a patient, preferably without the use of adhesive or needles.

The anchoring system includes an anchor pad and a retainer mounted on the anchor pad. The retainer is attached to the anchor pad which includes adhesive for attaching the pad to the patient's skin at the puncture site. A medical article is secured within the channel by capturing and holding a radially extending element on the medical article. One of the notable features of this system is that correct positioning of the catheter relative to the anchoring member can be achieved. However, once the catheter is secured at the site of insertion, there is no cover or protective structure that will prevent the accidental dislodgement of the catheter from the patient's skin.

Also known and considered relevant are U.S. Pat. Nos. 6,673,046, 6,770,055, D492,411, 6,689,104, 6,663,600, 6,582,403, 6,572,588, 6,551,285, D470,936, 6,491,664, 6,447,485, 6,428,516, 6,428,515, 6,413,240, 6,361,523, 6,290,676, 6,283,945, 6,224,571, 6,213,979, 6,132,398, 6,117,163, D425,619, 5,947,931, 5,941,263, D404,815, 5,855,591, 5,833,667, 5,827,230, 5,800,402, 5,702,371, 5,693,032, D377,831, D335,926, 5,238,010, 5,167,240, 3,528,416, 3,194,235, 3,900,026, 3,901,226, and 6,526,981, and WIPO Patent Publ. No. 93/06876.

Thus, there is still a need for an improved catheter cover that protects against unintentional movement of the catheter.

### SUMMARY OF THE INVENTION

The catheter cover structure provided in accordance with the invention was designed to provide an easy way to secure a catheter to a patient's skin at or in the vicinity of a catheter puncture site, while maintaining hygienic precautions and avoiding premature adhesion to the skin surface. In the presently preferred embodiment, the catheter cover structure of the present as well as a portion of the catheter tubing extending away from the puncture site, and a perimeter defining a bearing surface for engaging the patient's skin and supporting the shield about the puncture site.

In another aspect of the invention, the cover further includes side portions disposed on adjacent opposing sides of the cavity of the central portion, the side portions each supporting on an outer surface retention means for holding a portion of the catheter tubing that extends from within the central cavity. Each side portion slopes from its skin engaging perimeter to the dome portion to permit objects hitting the cover to be deflected and the cover from being dislodged.

Yet another aspect of the invention is that the cover includes a first surface for adhesion to the patient's skin and a second opposing surface on which the retention means are mounted, wherein the central cavity of the cover is defined by an upstanding elongated portion extending across the lateral extent of the cover. In one version of the invention, the perimeter of the cover has a circular configuration. In another version of the invention, the perimeter of the cover has a multi-sided, somewhat diamond-shaped configuration.

In yet another aspect of the invention, the central portion of the cover further includes a chamber within which a cannula and needle is received and protected against accidental dislodgement. The chamber extends laterally or diametrically, depending on whether the cover has a substantially square shape or a substantially circular shape, respectively, from one side of the cover to the opposite side, and includes, at one end of the chamber wall, catheter engagement means extending across the chamber and having an opening therein sized to snugly receive the diameter of a catheter and a narrowed neck portion at a location remote from the upper side of the cover. The catheter tubing is secured within the cover by engaging the neck portion of the catheter engagement means about the catheter diameter. The neck portion of the cover, when the catheter tubing is held in the wall opening, forces a slight constriction of the catheter tubing (if the tubing is flexible or resilient enough to be capable of constriction) or engages the catheter tubing with a tight friction fit as the tubing passes the constriction.

In still another aspect of the invention, there is provided on the cover at regions remote from the chamber catheter resilient retainers to permit the slack in the end of the catheter extending outwardly from the chamber to be taken up by winding the protruding portion of the catheter around the perimeter of the cover and using the retainers to secure the protruding end against the cover.

In other contemplated embodiments, a protective cover configured to be attached to a patient is provided that helps prevent undesired movement of a catheter disposed between the cover and the patient's skin. The cover can include a rigid dome configured to receive a catheter and having a first aperture through which the catheter can be inserted. A fastener can be coupled to the dome, and configured to fasten the cover to the patient. The dome can further include a first set of detents extending from the dome and configured to restrict movement of the catheter with respect to the dome. As used herein, the term “detent” can include ribs, discs, tabs, and so forth, such that movement of the catheter with respect to the dome can be restricted.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a top perspective view of the cover of the present invention;

FIG. 2 shows a portion of a catheter secured to the cover shown in FIG. 1;

FIG. 3 is a view of the underside of the cover shown in FIG. 1;

FIG. 4 illustrates the combined catheter and cover secured to a patient's arm;

FIG. 5 is a cross-sectional view, taken along section line 5-5 in FIG. 4, of the cover of the invention secured to a patient's arm and also showing a catheter secured to the cover with the needle inserted in the patient's arm;

FIG. 6 is a top plan view of the cover of the present invention; and

FIG. 7 is a bottom plan view of the cover of the invention.

FIG. 8 illustrates a perspective view of another embodiment of a protective cover.

FIG. 9 illustrates a top view of the protective cover of FIG. 8.

FIG. 10 illustrate a side view of the protective cover of FIG. 8.

FIG. 11 illustrate a back view of the protective cover of FIG. 8.

FIG. 12 illustrates a bottom view of the protective cover of FIG. 8.

FIGS. 13-14 illustrate perspective views of yet another embodiment of a protective cover.

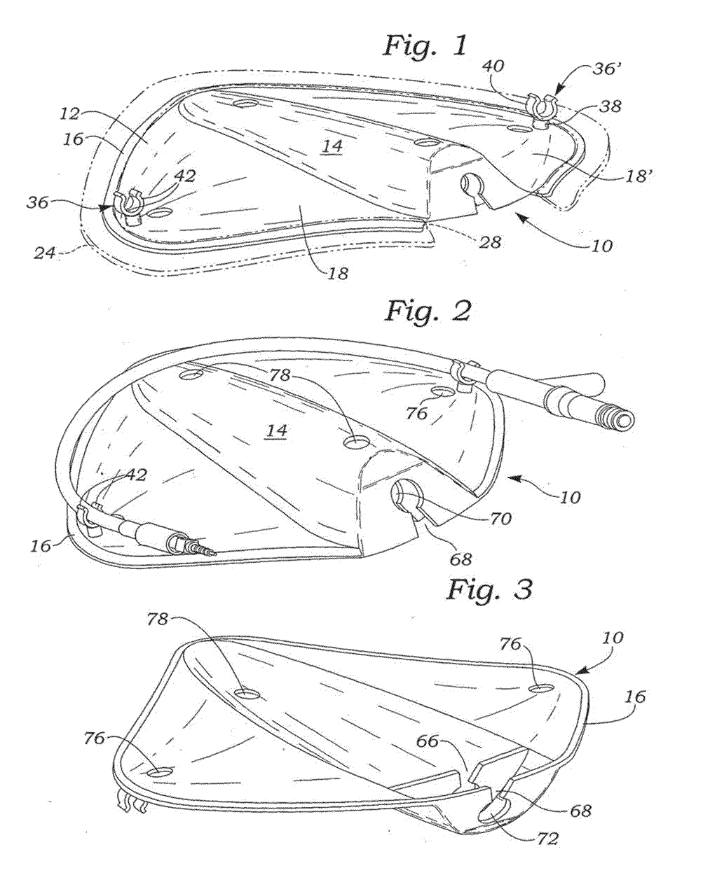

### DETAILED DESCRIPTION

One should appreciate that the disclosed techniques provide many advantageous technical effects including protecting an intravenous catheter from unintentional jarring or other movement while the catheter is inserted within a patient.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

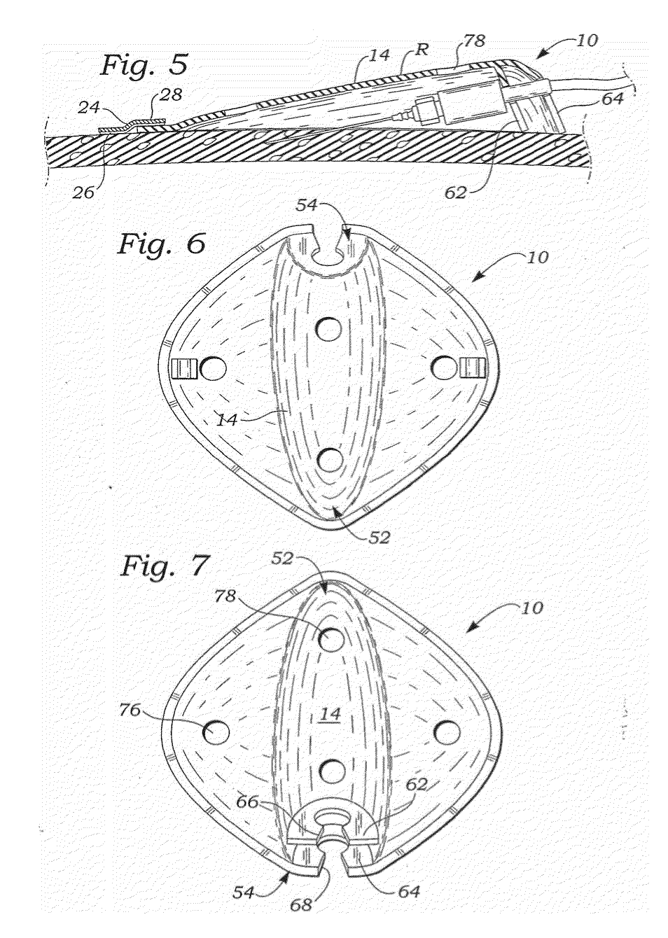

Referring now to the various figures of the drawing, the shield or cover 10 of the present invention is made of non-flexible material and includes a body 12 having a central chamber 14, an edge portion 16 extending about the perimeter of the body, and side portions 18, 18′ disposed on opposite sides of the chamber 14. The edge portion 16 is defined by a relatively narrow-width peripheral region arranged about the perimeter of the body, and preferably carries on its underside a layer of adhesive (see FIG. 5). Alternatively, the edge portion 16 of the cover 10 could be secured to the patient's skin by disposing about the outer perimeter of the edge portion a second member 24 having an adhesive layer on the bottom surface of a first region 26 thereof. A second inwardly located region 28 of the second member 24 is raised and captures and retains the edge portion 16 of the cover 10.

For the sake of description, the cover 10 includes a “front” portion and a “rear” portion (see FIGS. 6 and 7), and further has an inner surface that is disposed adjacent to the patient's skin when the cover is secured thereto, and an outer surface that is exposed to the ambient. The body 12 extends “longitudinally’ across the cover from the front portion 32 to the rear portion 34, and “laterally” between the side portions 18, 18′, each of the latter having slightly arcuate cross-sections. located at lateral extremities of the side portions of the first chamber are catheter tubing retention means 36, 36, each of which preferably comprise a stem 38 extending upwardly from the upper surface of a respective one of the side portions, and on each stem of which is supported a U-shaped member 40 including two opposing arms and a connecting bight portion. More preferably, the two arms 42, 42 are resiliently biased toward one another so that the tubing of the catheter is firmly gripped between the arms when the catheter is disposed therebetween (see FIG. 2).

Referring now to FIGS. 6, and 7, there is shown a central (second) chamber 14 extending substantially longitudinally across the cover 10. The second chamber includes a blunted nose portion 52 at the rear portion of the cover 10 and a mouth portion 54 at the front portion of the cover 10. The mouth portion 54 has a lateral extent, that is a “side-to-side” extent, and that is greater than the lateral extent of the nose portion 52. The central chamber 14 is defined by a U-shaped section of the cover that is raised above the upper surface of the first chamber of the cover 10. Preferably, the U-shaped section is disposed between the side portions 18, 18′. The U-shaped section of the second chamber 14 has a first height at the nose portion 52 that is nearly flush with the upper surface of the cover 10 and a second height at the mouth portion 54 that is substantially greater than the first height. Preferably, the height of the second chamber at the mouth portion is at least as great as the width of the second chamber at the mouth portion, and the length of the second chamber from front to back of the cover is at least twice as great as the height of the second chamber at the mouth portion thereof. More preferably, the length of the second chamber 14 from front to back of the cover is at least four times the height of the second chamber at the mouth portion thereof.

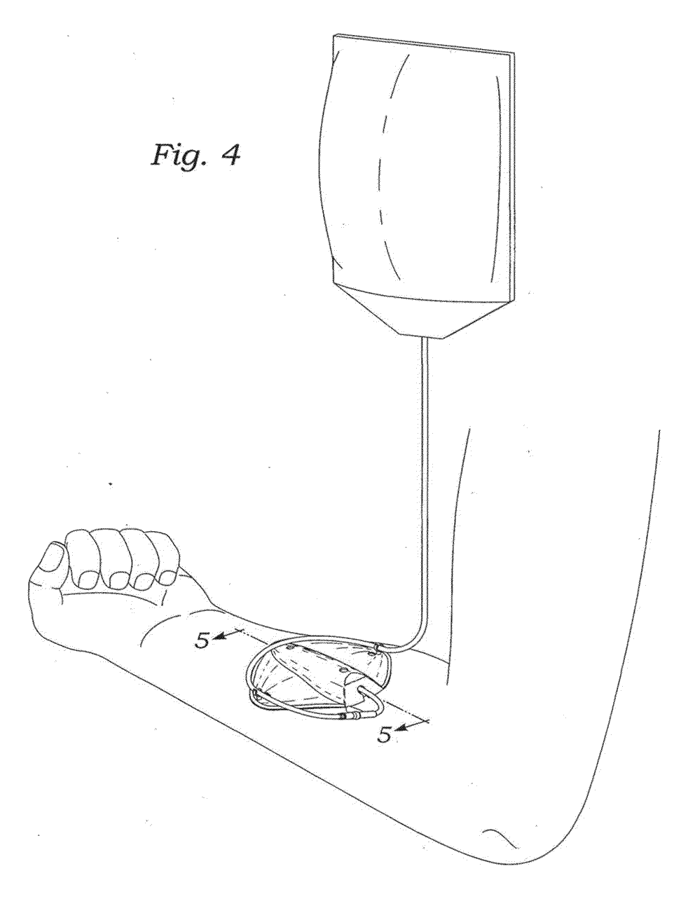

At the mouth portion of the second chamber 14 are inner and outer end walls 62, 64 (see FIGS. 3, 5 and 7). The end walls 62, 64 are arranged substantially perpendicular to the roof R of the chamber 14. Each of the inner and outer end walls are formed with slots 66. 68 that open toward the roof R of the second chamber, and a circular opening 70, 72 at the inner end of each of the respective slots. The slots are defined by opposing wall surfaces that converge toward the openings 70, 72, respectively, and the openings in the parallel inner and outer end walls 62, 64 are concentric and disposed adjacent to the roof R of the second chamber 14.

Ventilation holes 76, 76 are provided in side portions 18, 18′ of the body 12, and ventilation holes 78, 78 are provided at the top of the roof R of the second chamber 14. The side portions 18, 18′ secure and stabilize the second chamber 14 atop the puncture site.

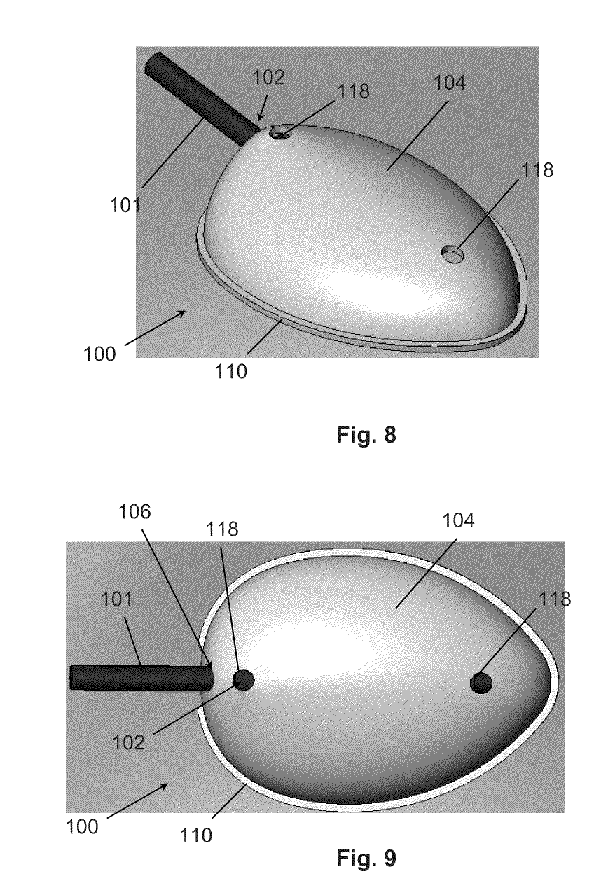

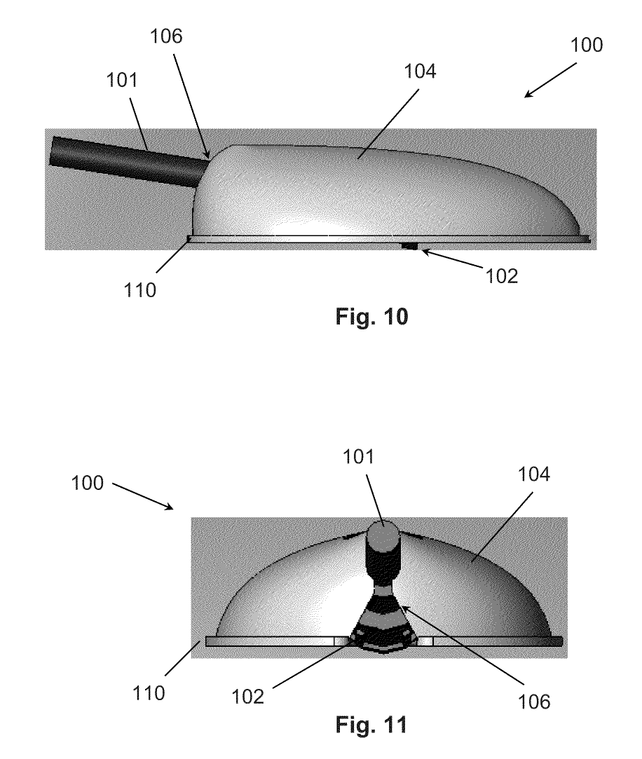

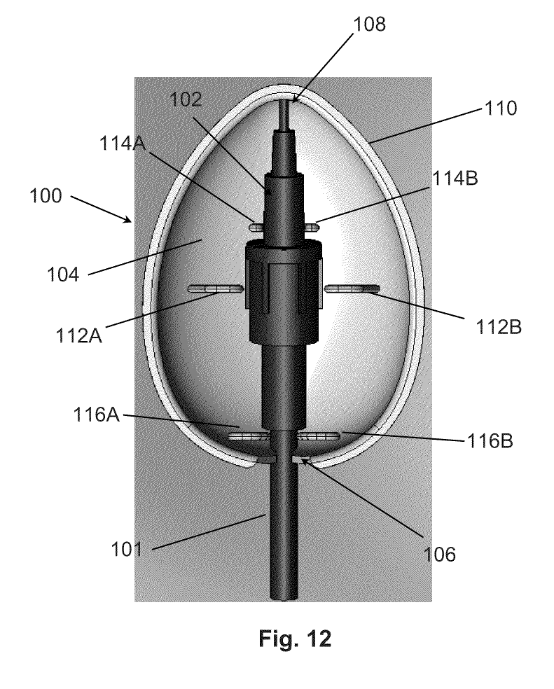

In FIGS. 8-12, a protective cover 100 is shown for an intravenous catheter 102, which is configured to attach to a patient (not shown). As used herein, the term “intravenous catheter” can include catheters, intravenous feed lines, as well as connecting hubs and other components typically fastened to a patient. Intravenous catheters include those commonly used in intravenous (IV) therapy such as peripheral IV lines, as well as central venous catheters (central lines) and other configurations used for intravenous therapies. The cover 100 can include a preferably rigid dome 104 configured to receive and store at least a portion of the catheter 102 within the curvature of the dome 104. In this manner, when the cover 100 is fastened to a patient, the catheter 102 can be disposed beneath the dome 104 and between the dome 104 and the patient's skin. Preferably, the cover 100 can include a first aperture 106 through which an intravenous feed line 101 or other portion of the catheter 102 can be inserted. The cover 100 could optionally include a second aperture (not shown) through which at least a portion of the catheter could be inserted. It is contemplated that at least a portion of a catheter tubing or needle 108 coupled to the catheter 102 will be disposed beneath the patient's skin while the cover 100 is coupled to the patient's skin, and the cover 100 can be used to prevent undesired or unintentional contact with the catheter tubing or needle 108.

The dome 104 can optionally include an outer lip 110, which is preferably disposed about an outer perimeter of the dome 104. The outer lip 110 can have a substantially flat surface, which allows the outer lip 110, and thereby the cover, to rest on the patient. However, it is alternatively contemplated that the outer lip 110 could have an at least partially curved surface.

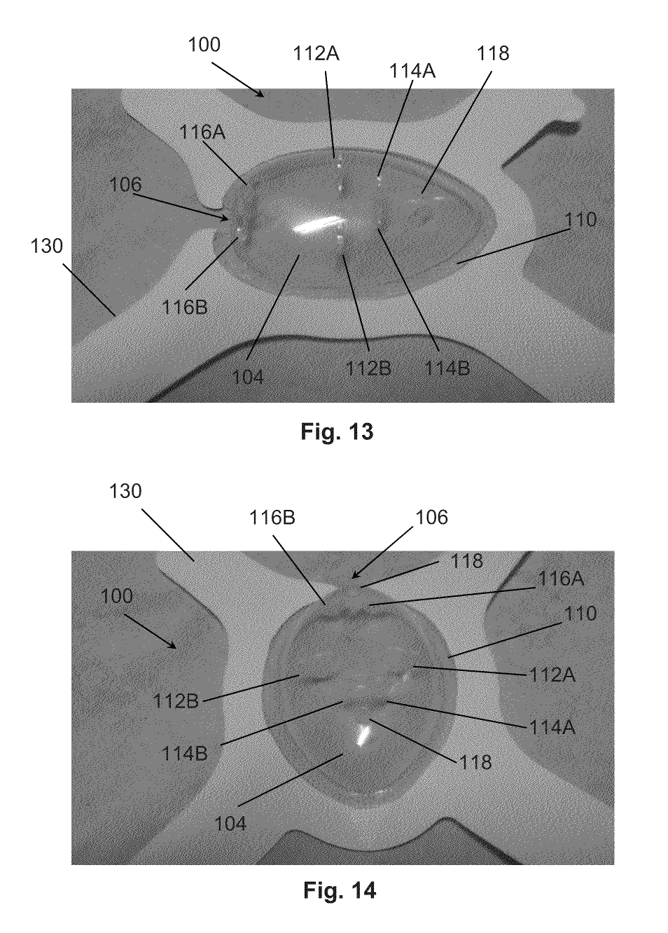

A bandage or other fastener shown in FIGS. 13-14 can be coupled to the dome 104 or the outer lip 110 of the cover 100. This advantageously allows the fastener to be used to fasten the cover 100 to the patient, and thereby secure and protect the catheter 102 from unintentional jarring or other movement. Preferred fasteners comprise an “X” or cross-arm shape, which helps prevent movement of the cover 100 when attached the patient, although any commercially suitable configuration could be used.

In preferred embodiments, the dome 104 includes a first set of detents 112A and 112B extending from the dome 104 and configured to restrict movement of the catheter 102 with respect to the dome 104. The dome 104 could also include a second set of detents 114A and 114B, and a third set of detents 116A and 116B, which further assist in preventing movement of the catheter 102 with respect to the dome 104. It is further contemplated that the first, second, and third sets of detents could comprise a single set of tabs. In addition, the specific number and arrangement of detents can vary depending upon the specific configuration of the catheter 102.

The dome 104 can further include one or more ventilation apertures 118, which are configured to ventilate an area beneath the dome 104 when the cover 100 is coupled to a patient. This advantageously helps air out the volume of air surrounding the catheter, and beneath the dome 104 and cover 100, and preferably can reduce moisture build-up beneath the cover 100.

In some contemplated embodiments, at least a portion of the dome 104 can include a photochromatic resin, which can thereby alter the color of the dome 104 over a predefined time period. This can be useful in determining the length of time the cover 100 and therefore the catheter has been in use, and the specific resin used can correspond with a recommended usage period for the catheter or cover 100. In this manner, when the dome 104 changes color, the patient or a nurse or other health practitioner can be readily alerted that the catheter or cover 100 should be changed.

It is further contemplated that the cover 100 can include one or more fluid sensors configured to detect a presence of fluid beneath the cover 100, or could alternatively include other types of sensors configured monitor other characteristics of the cover 100 including, for example, temperature. In addition, the cover 100 could include an RFID chip or other commercially suitable transmitter that is configured to transmit information concerning the cover 100 and/or patient to a receiver.

FIGS. 13-14 illustrate another embodiment of a protective cover 200 for an intravenous catheter 202. The cover 200 can include a bandage 230 or other fastener that can be used to attach the cover 200 to a patient via adhesive or other means. With respect to the remaining numerals in each of FIGS. 13-14, the same considerations for like components with like numerals of FIGS. 8-12 apply.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A cover for an intravenous catheter configured to attach to a patient, comprising:
a dome configured to receive a catheter, and comprising a first aperture through which the catheter can be inserted; and
wherein the dome comprises a first set of detents extending from the dome and configured to restrict a movement of the catheter with respect to the dome.

2. The cover of claim 1, further comprising an outer lip coupled to the dome, and disposed about a perimeter of the dome.

3. The cover of claim 1, wherein the dome is rigid.

4. The cover of claim 1, further comprising a fastener coupled to the dome, and configured to fasten the cover to a patient.

5. The cover of claim 4, wherein the fastener is coupled to at least a portion of an outer lip that is coupled to the dome, and wherein the outer lip is disposed between the fastener and the dome.

6. The cover of claim 1, wherein the dome further comprises ventilation apertures configured to ventilate an area beneath the dome when the cover is coupled to the patient.

7. The cover of claim 1, wherein the dome further comprises a photochromatic resin configured such that the dome changes color over a defined period.

8. The cover of claim 7, wherein the defined period is a recommended usage period for the catheter.

9. The cover of claim 1, further comprises a sensor coupled to the dome, and configured to monitor a presence of fluid beneath the dome.

10. The cover of claim 1, further comprising a second set of detents configured to restrict movement of the catheter with respect to the dome.

11. The cover of claim 4, wherein the fastener comprises an adhesive configured to allow for removably coupling of the dome to the patient.
